(19) United States
(12) Patent Application Publication
(10) Pub. No.: US 2007/0009566 A1
(43) Pub. Date: Jan. 11, 2007

(54) Method and apparatus for treating bacterial infections in devices

(76) Inventor: Naomi Balaban, Hopkinton, MA (US)

Correspondence Address: MERCHANT & GOULD PC, P.O. BOX 2903, MINNEAPOLIS, MN, 55402-0903, US

(21) Appl. No.: 11/395,294

(22) Filed: Apr. 3, 2006

Related U.S. Application Data
(60) Provisional application No. 60/667,939, filed on Apr. 4, 2005.

(51) Int. Cl. A61K 38/16; A61F 2/02

(52) U.S. Cl. 424/423; 514/2

(57) ABSTRACT

An apparatus and method for treating or preventing bacterial colonization of medical devices, or the host itself, comprise combining an RNAIII inhibiting peptide (RIP) with the medical device, optionally with an antibiotic. Further, RIP may be used in a lock technique, comprising adding sufficient solution comprising RIP to occupy a space within the device, which provides a high concentration of RIP at the actual or potential site of infection and prevents the space from filing with blood. The invention thus allows a clinician to maximize the amount of RIP and an antibiotic used to clean the medical device, while, at the same time, retaining the device within the host.

### CROSS REFERENCE TO RELATED APPLICATION

This application claims the benefit of U.S. Provisional Application Ser. No. 60/667,939, filed April 4, 2005, which is incorporated herein by this reference.

### FIELD OF INVENTION

The field of the present invention concerns an apparatus and a method for the treatment and prevention of bacterial biofilm formation and sepsis associated with use and/or implantation of medical devices.

### BACKGROUND

Microbial infection commonly attacks medical devices used in both clinical practice as well as scientific experiment. These devices often face microbial colonization both in vivo as well as prior to implantation. Moreover, surgical implantation of these devices may lead to sepsis and/or increasing susceptibility of microbial colonization in sites of injury.

Sepsis

Sepsis remains a leading cause of death, despite improvements in antimicrobial drugs and improved supportive care. Sepsis is associated with systemic inflammation, circulatory failure, and multiple organ dysfunction syndrome (“MODS”). Both Gram-positive microbes, such as Staphylococcus aureus, and Gram-negative bacteria can cause sepsis. The incidence of sepsis is currently on the rise. Angus et al., Crit. Care Med. 29: 1303-10 (2001). Gram-negative bacteria release lipopolysaccharide (“LPS”), or endotoxin, from their outer membrane, which elicits septic shock. By contrast, some Gram-positive bacteria cause septic shock by the release of enterotoxins, 23 to 29 kDa polypeptides in the bacterial superantigen protein family, such as toxic shock syndrome toxin-1 (TSST-1), and exotoxins, such as pyrogenic exotoxin A. Exotoxins are soluble substances that alter the normal metabolism of host cells with deleterious effects on the host, while enterotoxins are exotoxins that are specific for intestinal cells. De Kimpe et al., Proc. Nat'l Acad. Sci. USA 92: 10359-63 (1995); Kengatharan et al., J. Exp. Med. 188: 305-15 (1998); Llewelyn et al., Lancet Infect. Dis. 2: 56-162 (2002); Van Amersfoort et al., Clin. Microbiol. Rev. 16: 379-414 (2003).

Quorum Sensing and RNAIII—Inhibiting Peptide

Recent studies have evidenced the importance of quorum-sensing in the pathology of bacterial species including Vibrio cholerae, Pseudomonas aeruginosa, and Staphylococcus aureus. Quorum-sensing is a mechanism through which a bacterial population receives input from neighboring cells and elicits an appropriate response to enable itself to survive within the host. Balaban et al., Science 280: 438-40 (1998); Miller et al., Cell 110: 303-14 (2002); Hentzer et al., EMBO J. 22: 3803-15 (2003); Korem et al., FEMS Microbiol. Lett. 223: 167-75 (2003). In Staphylococcus, quorum-sensing controls the expression of proteins implicated in bacterial virulence, including colonization, dissemination, and production of multiple toxins involved in disease promotion. Some of these virulence factors are enterotoxins and toxic-shock syndrome toxin-1 (“TSST-1”), which act as superantigens to cause over-stimulation of the host immune system, causing excessive release of cytokines and inducing the hyper-proliferation of T cells.

In a quorum-sensing system in S. aureus, the effector quorum-sensing molecule RNAIII-activating peptide (“RAP”) phosphorylates “target of RNAIII-activating protein” (“TRAP”), a 21 kDa protein that is highly conserved among staphylococci. TRAP phosphorylation promotes bacterial adhesion and the downstream production of a regulatory RNA molecule termed RNAIII, which is responsible for toxin synthesis. Balaban (1998); Balaban et al., J. Biol. Chem. 276: 2658-67 (2001). An antagonist of RAP called RNAIII-inhibiting peptide (“RIP”) inhibits the phosphorylation of TRAP and thereby strongly inhibits the downstream production of virulence factors, bacterial adhesion, biofilm formation, and infections in vivo.

Biofilm has been shown to provide bacterial infections a very effective defense. Specifically, biofilms have demonstrated increasing resistance to antibiotics. Biofilms have a variety of attributes that contribute synergistically to the process of antibiotic resistance. These attributes include, but are not limited to, a lower growth rate, an exopolysaccharide matrix, a change in gene expression, an optimal three-dimensional structure, and the production of potentially resistant genes. Donlan et al., Clin Microbiol Rev. 15:167-193 (2002); Costerton et al., Science 284:1318-1322 (1999); and Fux et al., Trends Microbiol 13:34-40 (2005). In particular, matrix protects the microorganisms from the attack of antimicrobial therapy and from the immune system. The resistance to antimicrobial agents by the sessile bacterial communities is at the basis of many persistent and chronic bacterial infections and may be due to a number of factors, including the multilayer structure of biofilms and the unique genetic characteristics of bacteria in biofilms compared to those of planktonic cells. This phenomenon is not fully understood, and it is an area of active research.

Moreover, antibiotics used to treat biofilm infections in hospitals have to overcome the potential emergence of antibiotic resistance as well as the increased risk to the patient of developing allergies or adverse effects associated with antibiotics. Antibiotic efficacy also decreases as treatment time increases.

Unlike antibiotics, instead of killing bacteria, RIP inhibits bacterial cell-cell communication, rendering the bacteria more vulnerable to host defense mechanisms. See Balaban (1998); Balaban et al., Peptides 21: 1301-11 (2000); Gov et al., Peptides 22: 1609-20 (2001); Balaban et al., J. Infect. Dis. 187:625-30 (2003); Cirioni et al., Circulation 108: 767-71 (2003); Ribeiro et al., Peptides 24: 1829-36 (2003); Giacometti et al., Antimicrob. Agents Chemother. 47: 1979-83 (2003); Balaban et al., Kidney Int. 23: 340-45 (2003); Balaban et al., Antimicrob. Agents Chemother. 48: 2544-50 (2004); Dell'Acqua et al., J. Infect. Dis. 190: 318-20 (2004). In U.S. Pat. No. 6,291,431 to Balaban et al., the inventor has more fully described RIP's interference known as quorum sensing. RIP has had very impressive results including demonstrating strong activity against adhesion and virulence of S. aureus and S. epidermidis (22-24). Balaban et al., Science 280:438-440 (1998); and Balaban et al., J. Biol Chem 276:2658-2667 (2001).

Device Colonization by Bacteria

Bacteria colonize devices through various means. For example, the bacteria on a host's skin may colonize a device's exterior. Alternatively, bacteria may colonize a device through the device's interior. For example, many devices, such as catheters, have lumens, which provide the bacteria an ample colonization area.

After colonization, the bacteria then proceed to develop a biofilm structure. Specifically, as mentioned above, antibiotic therapy and prophylaxis aimed at killing the bacteria often fail to eradicate the infection due to the increased resistance of the biofilm-encased bacteria. Moreover, bacteria have evolved into various strains resistant to antibiotics such as methicillin and vancomycin, which further limits currently available therapeutic approaches.

Generally colonization and biofilm formation may occur within three days of catheterization. According to the Infectious Diseases Society of America (“IDSA”), the most common organisms responsible for causing catheter-related infections are gram-positive bacteria, such as coagulase-negative staphylococci and Staphylococcus aureus. Other organisms that can cause catheter-related infections include aerobic gram-negative bacilli and Candida albicans. The IDSA has published clinical guidelines on the treatment of catheter-related infections. For the treatment of nontunneled CVC-related bacteremia, removal of the catheter and treatment with systemic antibiotics or antifungal agents, depending on the organism, is recommended.

Treatment recommendations for device-related bacteremia differ somewhat. Often, device removal and systemic antibiotic treatment are often recommended. Unfortunately, because of the expense, time and difficulty of finding new access sites, insertion of a new device is not always desirable.

To combat bacterial colonization, device manufacturers have bonded various devices with antimicrobial substances. For example, manufacturers have pre-coated devices with antibiotics. McConnell et al., Clin Infect Dis. 37: 65-72 (2003); and Marciante et al., Am J Infect Control 31:1-8 (2003). Various antimicrobials and antiseptics also have been used. Unfortunately, some researchers have reported anaphylactic reactions in some patients. Hannan et al., Anaesthesia 54: 868-872 (1999). For these reasons, attention has turned to the in situ treatment of colonized devices or the lock technique (also known as intraluminal therapy). This technique involves the instillation of a concentrated antimicrobial solution into a colonized medical device, in a volume chosen to fill the lumen. This technique provides for high concentrations of antimicrobial agents at the site of infection combined with the low incidence of antibiotic toxicity.

Despite the suggested treatment regimen, the recurrence of infection is high. Biofilm bacteria can usually survive antibiotics at concentrations 1,000 to 1,500 times higher than antibiotic concentrations used to treat bactericidal planktonic bacteria. Donlan et al., Clin Microbiol Rev. 15:167-193 (2002); Costerton et al., Science 284:1318-1322 (1999); and Fux et al., Trends Microbiol 13:34-40 (2005). Various anti-biofilm strategies directed at detection and disruption of adherent bacteria are the focus of intense research.

### SUMMARY OF THE INVENTION

The instant invention teaches a unique, novel and useful apparatus and multiple methods for treating and preventing bacterial colonization of medical devices as well as in the host itself. In particular, the device and/or the host itself is at risk of infection by Gram-positive bacteria, such as Streptococcus ssp, including S. aureus and S. epidermidis, or an antibiotic resistant strain thereof. In other embodiments, the pathogen may be Listeria spp, including L. innocua, and L. monoctogenes, Lactococcus spp, Enterococcus spp, Escherichia coli, Clostridium acetobtylicum, and Bacillus spp, including B. subtilus, B. anthracis, and B. cereus or an antibiotic resistant strain thereof. The combination of RIP with a medical device alone and/or with an antibiotic dramatically reduces bacteria. Essentially, RIP interferes with the ability of the bacterium communication mechanism and thereby limits the bacterium's ability to form a strong biofilm. This reduces the bacteria's ability to secrete toxins. As an alternative embodiment, antibiotics can be added as well, thereby increasing the eradication of the bacteria on the device.

Another aspect of the invention centers on the technique used to combine RIP with the target device and the antibiotic to salvage the device. The device and a lock solution are intended to be used to maintain the device patency. For in vivo purposes, the solution component acts by physically occupying space within the device catheter and exerting pressure on the host's circulating blood, as applicable. In this way, the blood does not back fill into the device and clot. The lock technique provides an elegant method to maximize the concentration of solution within a particular device. For example, a technician can infuse solutions into the lumen of, for example, a catheter (or any other device with a lumen), using doses approximately 100 to 1,000 times higher than what is given systemically. As per standard guidelines, the solution—whether an antibiotic or RIP—should remain in the device for a period of time. In one embodiment, for example, RIP may stay in the device for thirty minutes, while a technician may then follow by letting the antibiotic sit in the device for an hour; however, again, these time measurements merely reflect a single embodiment and nearly any other amount of time may be used, although possibly with varying results.

Moreover, the lock technique can be used in vivo to maximize the treatment of the bacteria both on the device and/or in the host, without requiring a clinician to destroy the integrity of the inserted device. As noted above, one of the problems with bacteria colonization has been the required removal of a medical device for treatment of the bacterial infection. This invention illustrates a method to allow the clinician to maximize the amount of RIP and antibiotic to be used to cleanse the a medical device, while, at the same time, retaining the device within the host.

Device manufacturers also can easily pre-treat devices by infusing the particular device with RIP prior to a procedure. In one embodiment of the invention, a central venous catheter (“CVC”) can be combined with RIP prior to treatment. As the examples shown herein provide, this greatly limits bacteria's ability to form biofilm and enhances the efficacy of the antibiotics.

Simply put, the instant invention demonstrates a new approach to treating and preventing an invidious problem plaguing the medical and scientific communities.

### BRIEF DESCRIPTION OF THE FIGURES

FIG. 1 depicts a standard CVC.

FIG. 2 depicts a syringe that is used in one embodiment of the invention to infuse or remove the RIP from the CVC.

### DESCRIPTION OF SPECIFIC EMBODIMENTS

Description of Catheter and Syringe

For the instant invention, we provide a brief overview and description of a typical CVC 10 and syringe 50 that can be used in procedures involving the instant invention; however, in no way is this description meant to be limiting. Instead, this description is provided merely as an example as there are many other types of CVCs and/or catheters in general, as well as syringes, that can easily be substituted for the tools described herein. The inventors present these examples to provide a more detailed description of one embodiment of the instant invention.

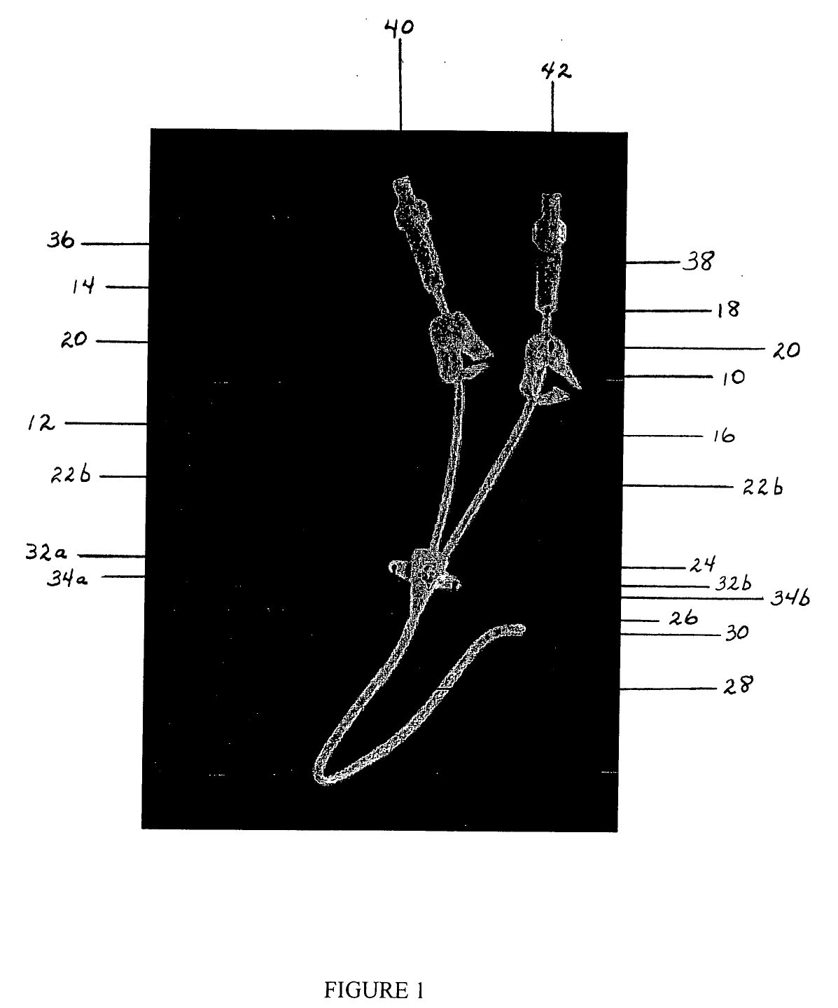

Referring to FIG. 1, a central venous catheter system, seen generally at 10, is shown. The catheter 10 includes a source tube 12, terminating in a fitting 14, which can be either male or female. Also, the catheter 10 has a return tube 16 that ends in a fitting 18, which also can be either male or female. Each tube includes a valve 20 that controls the flow of liquid through the catheter tubes.

The source tube 12 and the return tube 16 each include a lumen 22a and 22b. The source tube 12 and the return tube 16 are held in a connector manifold 24 that tapers in a direction away from the source tube 12 and the return tube 16, finally ending in a in a single exit point 26 from the manifold. The manifold 24 (the internal workings of which are not shown herein) establishes the respective pathways for the source tube 12 and the return tube 16 as the fluid drains through the respective lumens 22a and 22b and exits the end point 26 into the catheter body 26. The catheter body 26 also includes its own lumen 28 through which fluid passes and exits into or out of the host through the end port 30. The end port 30 may drain into or pull fluid from any number of areas of the host including, for example, the superior vena cava in humans.

While not a requirement in all central venous catheters, in this particular embodiment, the manifold 22 includes a suture anchor 32a and 32b on each side of the manifold 22. The anchors 32a and 32b are used for suturing the catheter 10 to a patient pursuant to general central venous catheter operating principles. The anchors 32a and 32b in this embodiment of the catheter 10 also include suture holes 34a and 34b for securing the catheter 10 to the host, however, this is only one example of a catheter. Clinicians often anchor catheters to hosts through other means as well, including, for example, using surgical tape and other adhesives.

As seen in FIG. 1, the fitting 14 is connected to a fluid tube 36, which has fluid 38, which, in the preferred embodiment is RIP and then heparin. The fitting 18 also includes a fluid tube 38, which is where a clinician would obtain a “return.” In other words, the clinician would draw fluid from the host (for example, to make sure that the passage way to/from the superior vena cava remains unobstructed). The fluid tubes 36 and 38, each have a respective end 40 and 42 that has a female mating in one embodiment, but, as is understood in the art, may include any type of mating. The ends 40 and 42 will mate with the vessel (not shown) carrying or receiving the fluid to be infused into or drained from the catheter 10 and/or the host.

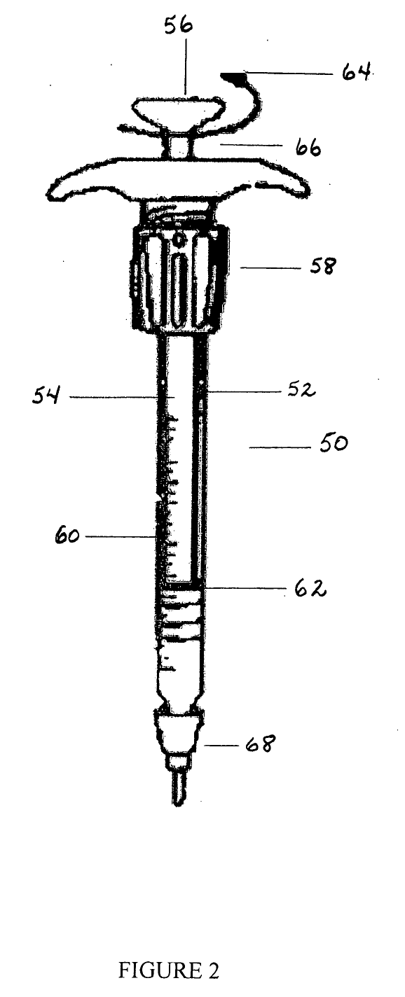

FIG. 2 includes a syringe 50. The syringe 50 is used to infuse the source tube 12 or remove fluid from the return tube 18. The syringe 50 includes a cylindrical housing 52 with an internal wall 54 and a plunger 56. The plunger 56 and the cylindrical housing 52 are connected through a cap 58, that mates with the top of the cylindrical housing (not shown). The syringe 50, also includes measuring notches 60 to determine the amount of fluid within the cylindrical housing 52. The plunger 56 has a flat end 62 that compresses the liquid contents within the cylindrical housing 52. The flat end 62 extends within the cylindrical housing 52 and seals with the internal wall 54. The plunger 56 has a fully retracted position 64 and a fully inserted position 66. In the fully inserted position 66, the plunger 56, by way of the flat end 62, expels the liquid within the cylindrical housing 52 through the syringe tip 68. In the fully retracted position 64, if the syringe 50 is inserted into a host, the cylindrical housing 52 becomes filled with liquid.

RNAIII-Inhibiting Peptides of the Invention

The quorum-sensing inhibitor RIP does not affect bacterial growth but reduces the pathogenic potential of the bacteria by interfering with the signal transduction that leads to production of exotoxins. RIP blocks toxin production by inhibiting the phosphorylation of its target molecule TRAP, which is an upstream activator of the agr locus. By contrast, the mechanism of action of antibiotics and/or antimicrobial peptides in general is to kill the bacterium. Because RIP and antibiotics act by different mechanisms, the two can act synergistically to treat bacterial infections.

The RIP polypeptide may comprise five contiguous amino acids of the sequence YX₂PX₁TNF, where X₁ is C, W, I or a modified amino acid, and X₂ is K or S; or amino acids having a sequence that differs from the sequence YX₂PX₁TNF by two substitutions or deletions, where X₁ is C, W, I or a modified amino acid, and X₂ is K or S. In one embodiment, the RIP does not consist of the sequence YSPX₁TNF, where X₁ is C, W, I or a modified amino acid. Alternatively, the RIP may comprise amino acids having a sequence that differs from the sequence YX₂PX₁TNF by one substitution or deletion, where X₁ is C, W, I or a modified amino acid, and X₂ is K or S. In various other embodiments, the RIP comprises the amino acid sequences YKPX₁TNF, where X₁ is C, W, I or a modified amino acid; the amino acid sequence IKKYX₂PX₁TNF, where X₁ is C, W, I or a modified amino acid and X₂ is K or S; or one of the sequences PCTNF, YKPITNF, or YKPWTNF. Consequently, in the various embodiments, RIP may contain differing amounts and types of amino acids in the polypeptide chain.

Biofilm Reduction and Synergistic Antibiotic Effect on Devices

Bacteria that attach to surfaces aggregate in a hydrated polymeric matrix of their own synthesis to form biofilms. Formation of these sessile communities and their inherent resistance to antimicrobial agents are at the root of many persistent and chronic bacterial infections. See Costerton et al., Science 284: 1318-22 (1999). Biofilms develop preferentially on inert surfaces such as devices; however, they can also form on living tissues. Biofilms grow slowly, in one or more locations, and biofilm infections are often slow to produce overt symptoms. Sessile bacterial cells release antigens and stimulate the production of antibodies, but the antibodies are not effective in killing bacteria within biofilms and may cause immune complex damage to surrounding tissues. Even in individuals with excellent cellular and humoral immune reactions, biofilm infections are rarely resolved by the host defense mechanisms. As will be discussed in greater detail below, antibiotic therapy typically reverses the symptoms caused by planktonic cells released from the biofilm, but fails to kill the biofilm. For this reason biofilm infections typically show recurring symptoms after cycles of antibiotic therapy, until the sessile population is surgically removed from the body. It is therefore preferable to prevent biofilm formation and/or reduce it once it has formed.

The apparatus and method of the present invention are useful in the treatment of bacterial infection and/or disease associated with biofilms for medical devices and, in one embodiment of the instant invention, a CVC. For example, the use of RIP infused in a CVC will reduce the risk that the implanted device will develop a biofilm. Moreover, in one embodiment, using the lock technique to infuse the CVC then adding an antibiotic will diminish the bacterial disease. Consequently, RIP-impregnated CVC's reduce biofilm and enhance the efficacy of the antibiotic lock technique for the salvage of a contaminated device and/or host.

### EXAMPLE

As an example to demonstrate the use of a RIP-impregnated CVC to enhance the efficacy of the antibiotic to salvage the CVC in one possible embodiment of the instant invention, we present the following example.

Materials and Methods

Organisms: The inventors chose the S. aureus strain Smith diffuse (SD). This is a highly encapsulated, slime producing strain with exopolysaccharides which are antigenically identical to many clinical S. aureus strains tested.

Antibiotics: While any antibiotic, produced synthetically or by an organism, which has the capacity to inhibit the growth of or to kill other microorganisms could be used, including, for example, daptomycin, in this embodiment of the instant invention, the inventors chose the following antibiotics to combine with RIP to demonstrate the synergistic effect: Vancomycin, ciprofloxacin and imipenem. The inventors diluted these antibiotics in accordance with manufacturers′ recommendations. Solutions were made fresh on the day of assay or stored at −80° C. in the dark for short periods. The concentration range assayed was 0.25-1,024 μg/ml.

Synthetic peptides: The amide form of RIP (YSPWTNF-NH2) was synthesized. RIP was then purified by HPLC to 99%. It was dissolved in distilled water (“H₂O”) at 20 times the required maximal concentration. The solution was made fresh on the day of assay and/or stored at −80° C. in the dark for short periods, as necessary.

Animals: The inventors used adult male Wistar rats (weight range, 250 to 300 g) (n=12/experimental group). All animals were housed in individual cages under constant temperature (22° C.) and humidity with 12-hours light/dark cycle, and had access to chow and water ad libitum throughout the study, The environment was temperature and humidity controlled, with lights on and off at 06:30 AM and 06:30 PM.

Adherent biofilm formation for susceptibility testing: To develop a biofilm, 50 μL of Triptic Soy broth (“TSB”) containing 106 CFU/mL of bacteria was added under aseptic conditions to each well of a tissue culture-treated polystyrene 96-well plate containing 150 μL of TSB-2% glucose. After 24 hours of incubation at 37° C., the growth medium was discarded and each well was washed three times with phosphate-buffered saline (“PBS”) under aseptic conditions to eliminate unbound bacteria. To evaluate the formation of adherent biofilm, the remaining attached bacteria were fixed with 0.2 mL of 99% methanol per well, and after 15 min, plates were emptied and left to dry. Then, the plates were stained for 5 minutes with 0.2 mL of 2% crystal violet per well for Gram staining the bacteria. Excess stain was rinsed off by placing the plate under running tap water. Stepanovic et al., J Microbiol Methods 40:175-179 (2000); and Christensen et al., J Clin Microbiol 22:996-1006 (1985). The plates were air dried. The dye bound to the adherent cells was solubilized with 0.2 mL of 33% (v/v) glacial acetic acid per well. The optical density (“OD”) of each well was determined photometrically at 570 nm by using the MR 700 Microplate Reader (Dynatech Laboratories, Guernsey, UK) and expressed OD. The blank (negative control) was determined for every plate measuring the optical density of a well filled with PBS solution. The cut-off OD for the microtiter-plate test was defined as three standard deviations above the mean OD of the negative control. The same experiment was performed two times: (i) with and (ii) without addition of 10 μg of RIP in a total volume of 10 μL MH broth into each well. In this embodiment of the instant invention, each test was performed in triplicate.

Susceptibility testing by classical method: Both for experiment (i) and (ii) as set forth in the preceding section, the minimal inhibitory concentration (“MIC”), or the lowest concentration of antibiotic sufficient to inhibit bacterial growth when tested in vitro, was determined using a microbroth dilution method with Mueller-Hinton (“MH”) broth and an initial inoculum of 5×10⁵ cfu/mL, according to the procedures outlined by the National Committee for Clinical Laboratory Standards. National Committee for Clinical Laboratory Standards, Fifth Edition, Approved Standard M7-A5, Wayne, Pa. (2001). Polystyrene 96-well plates were incubated for 18 hours at 37° C. in air. The MIC was taken as the lowest drug concentration at which observable growth was inhibited. The minimum bactericidal concentration (“MBC”), or the lowest concentration of antibiotic sufficient to kill a bacteria after removal of the drug, was taken as the lowest concentration of each drug that resulted in more than 99.9% reduction of the initial inoculum. In addition, to investigate the effect of RIP pre-treatment on bacterial antibiotic susceptibility, MIC and MBC were determined after pre-treatment of plates for 30 minutes with 10 μg of RIP in 10 μL MH broth/well. In this embodiment of the instant invention, each experiment was performed in triplicate.

Susceptibility testing with adherent cells: In this embodiment of the invention, the above described experiments were repeated with adherent bacteria cells. Biofilms (as prepared as described previously) were washed with PBS in order to discard unbound bacteria. Subsequently, serial twofold dilutions of antibiotics in MH broth were added to wells containing adherent organisms. The polystyrene plates were incubated for 18 hours at 37° C. in air. As previously, the MIC was taken as the lowest drug concentration at which observable bacteria growth was inhibited. To determine the MBC, the MH broth containing antibiotics was removed from each well and replaced with antibiotic-free MH broth. The plates were then incubated again for 18 hours at 37° C. in air. The MBC was taken as the lowest concentration of each drug that resulted in no bacterial growth following removal of the drug. The same experiments were repeated 30 minutes after the addition of 10 μg of RIP in a total volume of 10 μL MR broth into each well. As before, in this embodiment of the instant invention, all experiments were performed in triplicate.

Experimental Design in the Animals

Preparation of inoculum: The strain of bacteria was grown in a brain-heart infusion broth. When bacteria were in the log phase of growth the suspension was centrifuged at 1000 g for 15 minutes, the supernatant was discarded, and the bacteria were suspended and diluted into sterile saline to achieve a concentration of approximately 1×10¹⁰ CFU/ml.

Rat central venous catheter (CVC)—associated infection model: The rats underwent catheterization. Briefly, a silastic catheter was inserted into the jugular vein and was advanced into the superior vena cava. The proximal portion of the catheter was tunneled subcutaneously to exit in the midscapular space. A rodent restraint jacket was used to protect the catheter and to allow access to it. Twenty-four hours after CVC placement, blood cultures were obtained from the catheters to verify sterility. Rupp et al., J Antimicrob Chemother 47:705-707 (2001). Via the CVC, the rats were challenged with 1.0×10⁶ CFU of live S. aureus strain Smith diffuse. Six animals (control group 24 hours) were sacrificed 24 hours after the bacterial challenge to verify, by quantitative cultures, the presence of a bacterial infection. Contemporaneously, the lock technique was started for the other groups—the drugs were allowed to dwell for 1 hour in the CVCs and then were flushed with a heparin solution. The study included: (i) one control group (no CVC infection), (ii) one contaminated group that did not receive any antibiotic prophylaxis, (iii) one contaminated group that received RIP-impregnated CVC (by way of the lock technique), and (iv) six contaminated groups that received RIP-impregnated CVC (i.e. through the use of the lock technique) or no RIP-impregnated CVC plus ciprofloxacin, imipenem or vancomycin at concentrations equal to MBCs for adherent cells and 1.024 mg/ml in a volume of 0.1 ml that filled the CVC (i.e. through the use of the lock technique). Each experimental group included twelve animals.

Evaluation of treatment: To measure the in vivo results, quantitative peripheral blood cultures and quantitative cultures of the CVCs and surrounding venous tissues were performed.

On day seven of the experiments, the animals were killed. For quantitative peripheral blood cultures, peripheral blood was obtained by aseptic percutaneous transthoracic cardiac puncture and cultured on sheep blood agar plates. Plates were incubated at 37° C. for 48 hours and evaluated for the presence of the staphylococcal strain. The organisms were quantitated by counting the number of colony forming units (“CFU”) per plate. For quantitative cultures of the catheters and surrounding venous tissues, the location of the distal tip of the CVC in the superior vena cava was confirmed visually and the catheters and surrounding venous tissue were removed aseptically. The explanted catheters/venous tissues were placed in tubes containing 10 mL of PBS solution and sonicated for 5 minutes to remove the adherent bacteria. The solution was then cultured by performing serial dilutions (0.1 mL) of the bacterial suspension in 10 mM of sodium HEPES buffer (pH 7.2) to minimize the carryover effect and by culturing each dilution on blood agar plates. The plates were incubated at 37° C. for 48 hours and evaluated for the presence of the staphylococcal strains. The limit of detection for both methods was approximately <10 CFU/mL.

Statistical analysis: As seen in FIGS. 3 and 4, the MIC values are presented as the geometric mean of three separate experiments. Quantitative culture results from all groups are presented as mean±standard deviation. The bacterial counts obtained from peripheral blood, explanted CVCs and peripheral tissues were compared by using the Kruskal-Wallis test. Significance was accepted when the P value was ≦0.05.

Summarized Results

Susceptibility testing by classical method: According to the broth microdilution method recommended by the “NCCLS” (formerly known as the National Committee for Clinical Laboratory Standards), ciprofloxacin, imipenem and vancomycin exhibited MIC values of 1.00, 0.50 and 1.00 μg/ml, respectively. The pattern of susceptibility was confirmed by MBC determination, which showed for all agents values twofold higher. Similar results were obtained when the experiment was repeated 30 minutes after the addition of 10 μL of RIP into each well as seen in TABLE 1. Finally, RIP did not demonstrate any antibiotic activity against the strain (MIC>256 mg/l), as expected by its mechanism of action.

Susceptibility testing with adherent cells: The production of the biofilm was photometrically confirmed: the strain showed a mean OD_{570 nm} of 0.404±0.029. Originating from adherent single cells, the growing biofilm covered 5%±5% of the surface area after one day and increased to 50%±35% after seven days. The activity of the three antibiotics against the adherent bacteria was at least twofold lower than against the freely growing cell. In details, ciprofloxacin, imipenem and vancomycin exhibited against the adherent organisms MIC values 2.00, 1.00 and 2.00 μg/mL. In contrast, the MBC, showed an important increase for all agents used (eight-fold). Different data were obtained when the experiment was repeated 30 min after the addition of 10 μL of RIP into each well. In fact ciprofloxacin, imipenem and vancomycin showed MIC values of 1.00, 0.50 and 1.00 μg/mL. This pattern of susceptibility was confirmed by the MBC determinations, which showed values only twofold higher for all agents as seen in TABLE 1. Interestingly, in these series, slime production was much lower as confirmed by photometrical method (data not shown).

In vivo results: As shown in TABLE 2, all animals included in the control group (sacrificed after 24 hours) demonstrated evidence of CVC infection, with quantitative culture results showing 6.0×10⁶±1.7×10⁶ CFU/mL. Quantitative peripheral blood cultures obtained on day seven by transthoracic cardiac puncture evidenced that at concentration of MBC for adherent cells, ciprofloxacin, imipenem and vancomycin exerted weak antimicrobial activity, with results not significantly different if compare with untreated control. In contrast all other groups showed significant reduction in quantitative blood culture when compared to isotonic sodium chloride treated group (P<0.05). Significantly, only the groups treated with RIP plus the antibiotics at the highest concentrations showed no evidence of infection.

TABLE 1
Antimicrobial activity of ciprofloxacin, imipenem and vancomycin against
S. aureus strain Smith planktonic and adherent cells with or
without RIP pre-treatment.

| | Planktonic organisms | | Adherent organisms | |
|---|---|---|---|---|
| | MIC | | MIC | |
| Agent | (μg/ml) | MBC (μg/ml) | (μg/ml)^{b} | MBC (μg/ml)^{c} |
| Ciprofloxacin | 1.00 | 2.00 | 2.00 | 16.00 |
| Imipenem | 0.50 | 1.00 | 1.00 | 8.00 |
| Vancomycin | 1.00 | 2.00 | 2.00 | 16.00 |
| Ciprofloxacin* | 1.00 | 2.00 | 1.00 | 2.00 |
| Imipenem* | 0.50 | 1.00 | 0.50 | 2.00 |
| Vancomycin* | 1.00 | 2.00 | 1.00 | 2.00 |

*10 μg of RIP pre-treatment (in 10 μL MH broth/well).

TABLE 2
Efficacy of RIP, ciprofloxacin, imipenem and vancomycin in a rat model of CVC
infection induced by S. aureus Smith diffuse strain

| | | Quantitative |
|---|---|---|
| | | catheters/venous |
| | Quantitative blood | tissues |
| Treatment | culture (CFU/ml)^{a} | culture (CFU/ml)^{a} |
| Control group 24 h | — | 6.0 × 10⁶ ± 1.7 × 10⁶ |
| Isotonic sodium chloride solution | 9.6 × 10³ ± 1.9 × 10³ | 9.1 × 10⁷ ± 1.6 × 10⁷ |
| RIP | 3.4 × 10¹ ± 0.8 × 10^{1b} | 1.4 × 10³ ± 0.3 × 10^{3b} |
| Vancomycin 16.00 μg/ml | 4.2 × 10³ ± 1.5 × 10³ | 6.3 × 10⁶ ± 2.0 × 10⁶ |
| Vancomycin 1,024 μg/ml | 5.3 × 10¹ ± 1.1 × 10^{1bc} | 5.4 × 10⁵ ± 1.6 × 10^{5bc} |
| Ciprofloxacin 16.00 μg/ml | 6.5 × 10³ ± 1.7 × 10³ | 8.7 × 10⁶ ± 2.3 × 10⁶ |
| Ciprofloxacin 1,024 μg/ml | 6.6 × 10¹ ± 1.4 × 10^{1bc} | 7.4 × 10⁵ ± 2.1 × 10^{5bc} |
| Imipenem 8.00 μg/ml | 5.8 × 10³ ± 1.2 × 10³ | 6.9 × 10⁶ ± 2.1 × 10⁶ |
| Imipenem 1,024 μg/ml | 5.7 × 10¹ ± 1.2 × 10^{1bc} | 6.0 × 10⁵ ± 1.9 × 10^{5bc} |
| RIP plus Vancomycin 16.00 μg/ml | 2.6 × 10¹ ± 0.2 × 10^{1bc} | 1.2 × 10³ ± 8.3 × 10^{2bc} |
| RIP plus Vancomycin 1,024 μg/ml | <10^{bc} | 1.9 × 10¹ ± 0.2 × 10^{1bc} |
| RIP plus Ciprofloxacin 16.00 μg/ml | 4.9 × 10¹ ± 1.2 × 10^{1bc} | 3.4 × 10³ ± 0.3 × 10^{3bc} |
| RIP plus Ciprofloxacin 1,024 μg/ml | <10^{bc} | 3.1 × 10¹ ± 0.7 × 10^{1bc} |
| RIP plus Imipenem 8.00 μg/ml | 3.2 × 10¹ ± 0.8 × 10^{1bc} | 2.5 × 10³ ± 1.0 × 10^{3bc} |
| RIP plus Imipenem 1,024 μg/ml | <10^{bc} | 2.2 × 10¹ ± 0.5 × 10^{1bc} |

^{a}Mean ± S.D.
^{b}P < 0.05 versus the isotonic sodium chloride solution treated group.
^{c}P < 0.05 versus singly treated groups at concentration of MBC^{b}.

In this embodiment of the instant invention, similar results were obtained when the quantitative cultures were performed with the explanted catheters/venous tissues. Under these conditions, RIP and vancomycin at 1,024 μg/mL produced the greatest reduction in the bacterial numbers (1.9×10¹±0.2×10¹ CFU/ml) although no statistically significant difference was observed when compared to the other combined treated groups.

Summary of the Example Provided

As noted in the background, organisms that colonize the CVC originate either from the skin insertion site, migrating along the external surface of the device, or from the hub, due to manipulation by health care workers, migrating along the inner lumen. Thus, the recommended course of action for treatment of staphylococcal biofilm infections is to remove the infected device, treat the patient with rigorous antibiotic therapy, and reinsert a new device. Shorr et al., Chest 124:275-285 (2003) and Raad, Lancet 351:893-898 (1998).

In this embodiment of the instant invention, a quorum sensing inhibitor RIP was used as a way to protect the device from bacterial adherence and therefore increase the efficacy of the antibiotics, which work better on non adherent, planktonic bacteria. The results present herein showed the efficacy of RIP, vancomycin, ciprofloxacin and imipenem in the treatment of a CVC infection using the lock technique. As shown by the results, the antimicrobial agents were scarcely active against adherent bacteria, but were significantly enhanced by the presence of RIP. In fact, all antibiotics showed MICs and MBCs much lower than that obtained in absence of RIP. This example clearly demonstrated that the use of a RIP-impregnated CVC in combination with conventional antibiotics was associated with a significantly lower colonization rate as described in this embodiment of the instant invention. In fact, RIP and ciprofloxacin, imipenem and vancomycin at the highest concentrations tested produced a significant reduction of the staphylococcal infection of the device with a 5 log₁₀ decrease of the bacterial count compared with control. This reduction in colonization for quantitative catheters/venous tissues culture was statistically different between the two types of experiments performed with or without RIP.

All publications and patents mentioned herein are incorporated herein by reference to disclose and describe the specific methods and/or materials in connection with which the publications and patents are cited. The publications and patents discussed herein are provided solely for their disclosure prior to the filing date of the present application. Nothing herein is to be construed as an admission that the present invention is not entitled to antedate such publication or patent by virtue of prior invention. Further, the dates of publication or issuance provided may be different from the actual dates which may need to be independently confirmed.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. An apparatus comprising:
a host insert device for use in a medical or scientific procedure, having an exterior and a lumen, the host insert device adapted for insertion into a host; and
a pharmaceutical composition comprising an amount of polypeptide, the composition loaded within the lumen for prevention or treatment of bacteria biofilm formation occurring before, during or after the procedure whereby the composition can both treat and prevent bacteria biofilm formation of the lumen of the device.

2. The apparatus in claim 1 wherein the host insert device is selected from the group consisting of a catheter, stent, endoscope, IV, intrauterine device and a port.

3. The apparatus of claim 2 wherein the host insert device is a central venous catheter.

4. The apparatus in claim 3 wherein the host insert device is adapted for insertion into a human or an animal.

5. The apparatus in claim 1 wherein the polypeptide is RIP.

6. The apparatus in claim 1 wherein the amount of the peptide completely fills the lumen of the device.

7. The apparatus in claim 1 for use in a method for preventing or treating bacteria which is biofilm producing.

8. The apparatus in claim 7 for use in a method for preventing or treating bacteria where the bacteria is Staphylococcus epidermidis, Staphylococcus aureus or Bacillus anthracis.

9. A method for preventing bacteria biofilm formation in a device used in a medical or scientific procedure, the device having an exterior and a lumen, the method comprising:
selecting a device;
loading an amount of polypeptide into the device;
allowing the polypeptide to stand in the device for a period of time;
drawing out the polypeptide from the device; and
flushing the device.

10. The method of claim 9 further comprising the step of selecting a device from the group consisting of a catheter, stent, endoscope, IV, intrauterine device and a port.

11. The method of claim 9 further comprising the step of using a central venous catheter as the device in the procedure.

12. The method of claim 9 wherein the step of loading includes completely filling the lumen with polypeptide.

13. The method of claim 12 wherein the step of loading includes using a syringe.

14. The method of claim 9 further comprising the step of using RIP as the polypeptide.

15. The method of claim 14 wherein the step of selecting comprises selecting an amount of RIP sufficient to fill completely the lumen of the device.

16. The method of claim 9 wherein the polypeptide stands in the device for at least thirty minutes.

17. The method of claim 9 wherein the polypeptide is drawn from the device using the syringe.

18. The method of claim 9 wherein the device is flushed with a solution.

19. The method of claim 18 wherein the solution is herapin.

20. The method of claim 9 wherein the device is inserted into a host, which can be an animal or a human, prior to or during a medical or scientific procedure.

21. A method for treating a host and a device, the device being used in a medical or scientific procedure and having an exterior and a lumen, and the device having been inserted into the host, and bacteria having been detected in the host in the lumen of the device, the method comprising:
engaging the host to treat the bacteria, while maintaining the device's integrity within the host;
coupling a tube to the lumen;
loading the tube with an amount of polypeptide so that the polypeptide drains into the lumen;
allowing the polypeptide to stand within the device for a certain period of time;
drawing out the polypeptide from the device;
flushing the device; and
disengaging from the device and host so as not to disrupt the integrity of the device within the host.

22. The method of claim 21 further comprising the step of selecting a device from the group consisting of a catheter, stent, endoscope, IV, intrauterine device and a port.

23. The method of claim 22 further comprising the step of using a central venous catheter as the device in the procedure.

24. The method of claim 21 wherein the step of loading includes completely filling the lumen with polypeptide.

25. The method of claim 24 wherein the step of loading includes using a syringe.

26. The method of claim 21 further comprising the step of using RIP as the polypeptide.

27. The method of claim 21 wherein the polypeptide stands in the device for at least thirty minutes.

28. The method of claim 21 wherein the step of drawing out the polypeptide from the device is accomplished through a syringe.

29. The method of claim 21 wherein the device is flushed with a solution.

30. The method of claim 29 wherein the solution is herapin.

31. The method of claim 21 wherein the device is being inserted into a host which is an animal or a human.

32. An apparatus comprising:
a host insert device for use in a medical or scientific procedure, having an exterior and a lumen, the host insert device adapted for insertion into a host;
a composition comprising a polypeptide, the composition loaded within the lumen for prevention or treatment of bacteria biofilm formation occurring before, during or after the procedure; and
a pharmaceutical composition comprising an amount of an antibiotic, the composition loaded within the lumen with or separate from the polypeptide whereby the polypeptide can treat or prevent bacteria biofilm formation on the lumen of the device or the host and the antibiotic compositions can kill bacteria in the lumen of the device or the host.

33. The apparatus in claim 32 wherein the host insert device is selected from the group consisting of a catheter, stent, endoscope, IV, intrauterine device and a port.

34. The apparatus of claim 33 wherein the host insert device is a central venous catheter.

35. The apparatus in claim 34 wherein the host insert device is adapted for insertion into a human or an animal.

36. The apparatus in claim 32 wherein the polypeptide is RIP.

37. The apparatus in claim 32 wherein the amount of polypeptide is the amount necessary to fill completely the lumen of the device.

38. The apparatus in claim 32 wherein the bacteria is biofilm producing.

39. The apparatus in claim 39 for use in treating the bacteria, which is Staphylococcus epidermidis, Staphylococcus aureus or Bacillus anthracis.

40. The apparatus of claim 32 wherein the antibiotic is any chemical substance produced synthetically or by an organism which has the capacity to inhibit the growth of or to kill other microorganisms.

41. The apparatus of claim 40 wherein the antibiotic is selected from the group consisting of vancomycin, ciprofloxacin, daptomycin and imipenem.

42. The apparatus of claim 32 wherein the amount of antibiotic is the amount necessary to fill completely the lumen of the device.

43. A method for preventing bacteria biofilm formation in a device used in a medical or scientific procedure, the device having an exterior and a lumen, the method comprising:
selecting a device;
loading an amount of polypeptide into the device;
allowing the polypeptide to stand in the device for a period of time;
drawing out the polypeptide from the device;
flushing the device;
loading an amount of antibiotic into the device;
allowing the antibiotic to stand in the device for a period of time;
drawing out the antibiotic from the device; and
flushing the device.

44. The method of claim 43 further comprising the step of selecting a device from the group consisting of a catheter, stent, endoscope, IV, intrauterine device and a port.

45. The method of claim 43 further comprising the step of using a central venous catheter as the device in the procedure.

46. The method of claim 43 wherein the polypeptide is loaded into the lumen to fill the lumen.

47. The method of claim 46 wherein the polypeptide is loaded through use of a syringe.

48. The method of claim 43 further comprising the step of using RIP as the polypeptide.

49. The method of claim 48 further comprising the step of selecting an amount of RIP sufficient to fill completely the lumen of the device.

50. The method of claim 43 wherein the polypeptide stands in the device for at least thirty minutes.

51. The method of claim 43 wherein the polypeptide is drawn from the device using the syringe.

52. The method of claim 43 wherein the device is flushed with a solution.

53. The method of claim 52 wherein the solution is herapin.

54. The method of claim 43 wherein the device is inserted into a host, which can be an animal or a human, prior to or during a medical or scientific procedure.

55. The method of claim 43 further comprising the step of using a central venous catheter as the device in the procedure.

56. The method of claim 43 further comprising the step of selecting an antibiotic that is any chemical substance produced synthetically or by an organism which has the capacity to inhibit the growth of or to kill other microorganisms.

57. The method of claim 56 further comprising the step of selecting an antibiotic from the group consisting of vancomycin, ciprofloxacin, daptomycin and imipenem.

58. The method of claim 43 further comprising the step of filling the lumen with an amount of antibiotic necessary to fill completely the lumen of the device.

59. The method of claim 58 wherein the antibiotic is loaded through use of a syringe.

60. The method of claim 43 wherein the antibiotic stands in the device for at least one hour.

61. The method of claim 43 wherein the antibiotic is drawn from the device using the syringe.

62. A method for treating a host and a device, the device being used in a medical or scientific procedure and having an exterior and a lumen, and the device having been inserted into the host, and bacteria having been detected in the host in the lumen of the device, the method comprising:
engaging the host so as to stabilize the device, while maintaining the device's integrity within the host;
securing a tube attached to and directly working in concert with the lumen;
loading the tube with an amount of polypeptide so that the polypeptide drains into the lumen;
allowing the polypeptide to stand within the device for a certain period of time;
drawing out the polypeptide from the device;
flushing the device;
loading an amount of antibiotic into the device;
allowing the antibiotic to stand in the device for a period of time;
drawing out the antibiotic from the device;
flushing the device; and
disengaging from the device and host so as not to disrupt the integrity of the device within the host.

63. The method of claim 62 further comprising the step of selecting a device from the group consisting of a catheter, stent, endoscope, IV, intrauterine device and a port.

64. The method of claim 64 further comprising the step of using a central venous catheter as the device in the procedure.

65. The method of claim 62 wherein an amount of polypeptide is loaded into the lumen as is necessary to fill the lumen.

66. The method of claim 65 wherein the polypeptide is loaded through use of a syringe.

67. The method of claim 62 further comprising the step of using RIP as the polypeptide.

68. The method of claim 62 wherein the polypeptide stands in the device for at least thirty minutes.

69. The method of claim 62 wherein the polypeptide is drawn from the device using the syringe.

70. The method of claim 62 wherein the device is flushed with a solution.

71. The method of claim 70 wherein the solution is herapin.

72. The method of claim 62 wherein the device is being inserted into a host which is an animal or a human.

73. The method of claim 62 further comprising the step of selecting an antibiotic from the group consisting of vancomycin, ciprofloxacin, daptomycin and imipenem.

74. The method of claim 62 further comprising the step of filling the lumen with an amount of antibiotic necessary to fill completely the lumen of the device.

75. The method of claim 74 wherein the antibiotic is loaded through use of a syringe.

76. The method of claim 62 wherein the antibiotic stands in the device for at least one hour.

77. The method of claim 62 wherein the antibiotic is drawn from the device using the syringe.
